(19) United States
(12) Patent Application Publication
(10) Pub. No.: US 2014/0170766 A1
(43) Pub. Date: Jun. 19, 2014

(54) Aptamer Based Point-of-Care Test for Glycated Albumin

(71) Applicant: Epinex Diagnostics, Inc., Tustin, CA (US)

(72) Inventor: Henry J. Smith, Temecula, CA (US)

(73) Assignee: Epinex Diagnostics, Inc., Tustin, CA (US)

(21) Appl. No.: 14/069,544

(22) Filed: Nov. 1, 2013

Related U.S. Application Data
(63) Continuation of application No. 12/221,429, filed on Aug. 4, 2008, now abandoned.
(60) Provisional application No. 60/963,434, filed on Aug. 6, 2007.

(51) Int. Cl. G01N 33/53

(52) U.S. Cl. CPC G01N 33/5308
USPC 436/501

(57) ABSTRACT

Disclosed herein is a point-of-care or home use device for measuring the ratio of glycated albumin to total albumin in saliva and other body fluids. The ratio of glycated albumin to total albumin in saliva will provide an indication of the average amount of protein glycation that occurred over the preceding 2-3 week period. The test is performed using a disposable strip or cassette that contains the testing reagents and the results are measured in a measuring instrument that automatically reads, calculates and displays the final result. The results of tests performed over a period of time are stored in the instrument's memory and presented in a numerical or graphical format so that the individual's glycated albumin level can be monitored over time.

### CROSS-REFERENCE TO RELATED APPLICATIONS

This utility patent application claims priority to Provisional Patent Application Ser. No. 60/963,434 filed Aug. 21, 2007 entitled POINT-OF-CARE TEST FOR GLYCATED ALBUMIN.

### REFERENCES

LifeScan Inc. Product Brochure One Touch Brand Glucose Meter Product Insert.

Exocell Inc. Glycaben kit. Product insert.

Smith H. J. and Zaidi A. Rapid Test for Glycated Albumin WO 2005/031356 A1

Smith H. J. Rapid Test for Glycated Albumin in Saliva. U.S. patent application Ser. No. 11/140,306

Systematic Evolution of Ligands by Exponential Enrichment (SELEX). Gold L. et al U.S. Pat. No. 5,270,163

### FEDERALLY SPONSORED RESEARCH OR DEVELOPMENT

None

### BACKGROUND OF THE INVENTION

Diabetes mellitus or diabetes is a disease characterized by elevated levels of plasma glucose. Uncontrolled hyperglycemia is associated with increased risk of vascular disease including, nephropathy, neuropathy, retinopathy, hypertension, and death. There are two major forms of diabetes. Type 1 diabetes (or insulin-dependent diabetes) and Type 2 diabetes (or noninsulin-dependent diabetes). The American Diabetes Association has estimated that approximately 6% of the world population has diabetes.

The goal of diabetic therapy is to maintain a normal level of glucose in the blood. The American Diabetic Association has recommended that diabetics monitor their blood glucose level at least three times a day in order to adjust their insulin dosages and/or their eating habits and exercise regimen. However, glucose tests can only measure a point in time result and does not provide an overall assessment of glycemic control over a period of time.

To assess glycemic control over an extended period of time it is also recommended that hemoglobin A1c (glycated hemoglobin) testing be done 2-4 times a year. When blood proteins including hemoglobin are exposed to glucose over a period of time they become glycosylated and the degree of glycosylation is dependent on the average concentration of glucose and the length of time the proteins were exposed to the glucose. The level of glycated hemoglobin is also dependent upon the half-life of the hemoglobin molecule within the body. The net result is that measurement of glycated hemoglobin provides an estimate of the degree of glycosylation that occurred over the preceding 2-3 months.

It would be desirable to have a test that would provide an earlier indication of glycemic control to allow earlier therapeutic intervention. It would also be desirable to have a test that did not require the invasive process of obtaining a blood sample.

It would also be desirable to develop a simplified point-of-care assay that could be utilized in a doctor's office or by the patient at home.

### BRIEF SUMMARY OF THE INVENTION

This invention describes a non-invasive point-of-care method of measuring glycated albumin compared to total albumin using a saliva sample or other body fluid sample. Saliva albumin is derived from plasma albumin and saliva therefore would be expected to contain glycated and non-glycated albumin fractions. The ratio of glycated albumin to total albumin in saliva will provide an indication of the average amount of protein glycation that occurred over the preceding 2-3 week period.

Frequent monitoring of the individuals glycated albumin would provide an accurate assessment of overall effectiveness of glycemic control in the individual and allow earlier therapeutic intervention compared to the glycated hemoglobin test in current use.

The present invention describes the use of aptamers to develop a point-of-care device and/or home use device for measuring glycated albumin in saliva and other body fluids. The measuring device consists of two components: a disposable test cassette and a reusable measuring instrument.

### BRIEF DESCRIPTION OF THE DRAWINGS

FIG. 1A-B depicts an illustration of the disposable test strip containing the reagents and the placement of the components required to measure glycated and non-glycated albumin. FIG. 1A depicts an overhead view of the test strip. FIG. 1B depicts a side view of a test cassette.

FIG. 2. is an illustration of a fluorescence measuring instrument into which the test strip is inserted. The indicator agent used in the test strip is a fluorescing compound and the amount of fluorescence measured at the glycated albumin band region and at the non-glycated albumin band region is used to calculate the ratio of glycated albumin to total albumin in the sample.

FIG. 3. is an illustration of a spectrophotometer measuring instrument into which the test strip is inserted. The indicator agent used in the test strip is a colored compound and the amount of color measured at the glycated albumin band region and at the non-glycated albumin band region is used to calculate the ratio of glycated albumin to total albumin in the sample.

FIG. 4. is an illustration of a magnetic measuring instrument into which the test strip is inserted. The indicator agent used in the test strip are paramagnetic particles and the strength of the magnetic field measured at the glycated albumin band region and at the non-glycated albumin band region is used to calculate the ratio of glycated albumin to total albumin in the sample.

FIG. 5A-C depicts an illustration of a biosensor measuring instrument into which the test cassette is inserted. The indicator agent used in the test cassette is the electric potential when the glycated albumin binds to the glycated albumin electronic detector; and also the electric potential when the albumin binds to the albumin electronic detector. The ratio of glycated albumin to total albumin is then calculated. FIG. 5A depicts one embodiment of a biosensor cassette. FIG. 5B depicts one embodiment of a biosensor instrument. FIG. 5C depicts a second embodiment of a biosensor cassette.

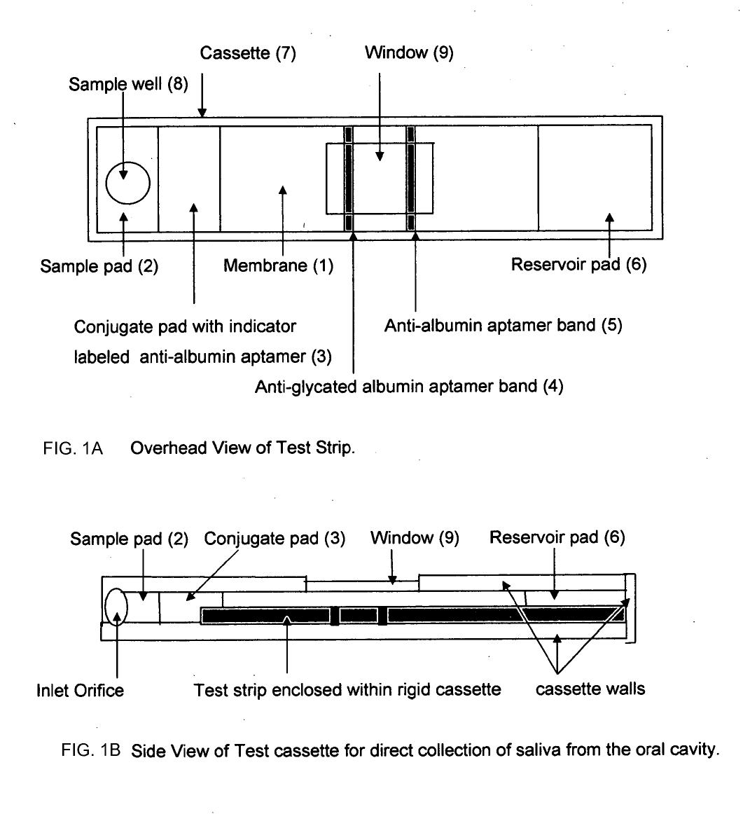

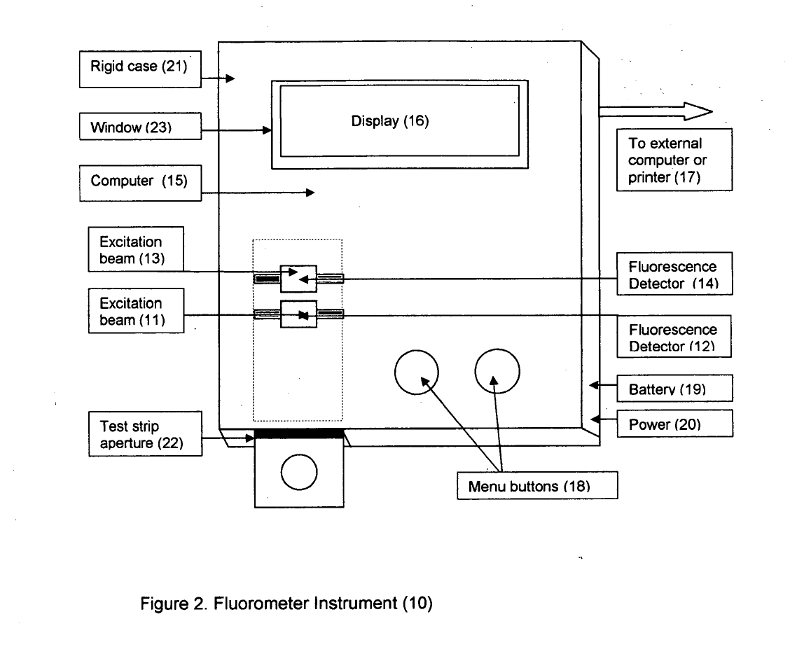

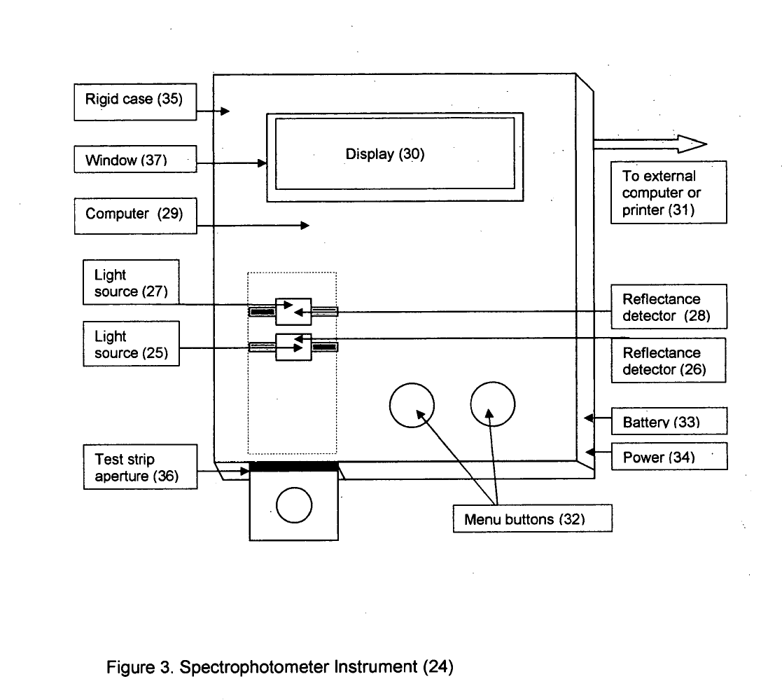

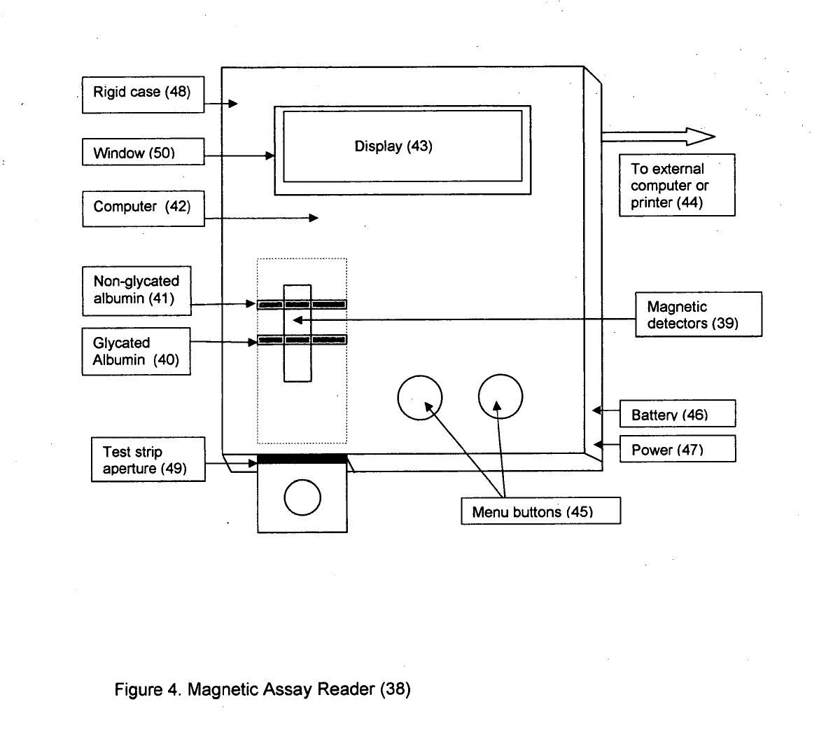

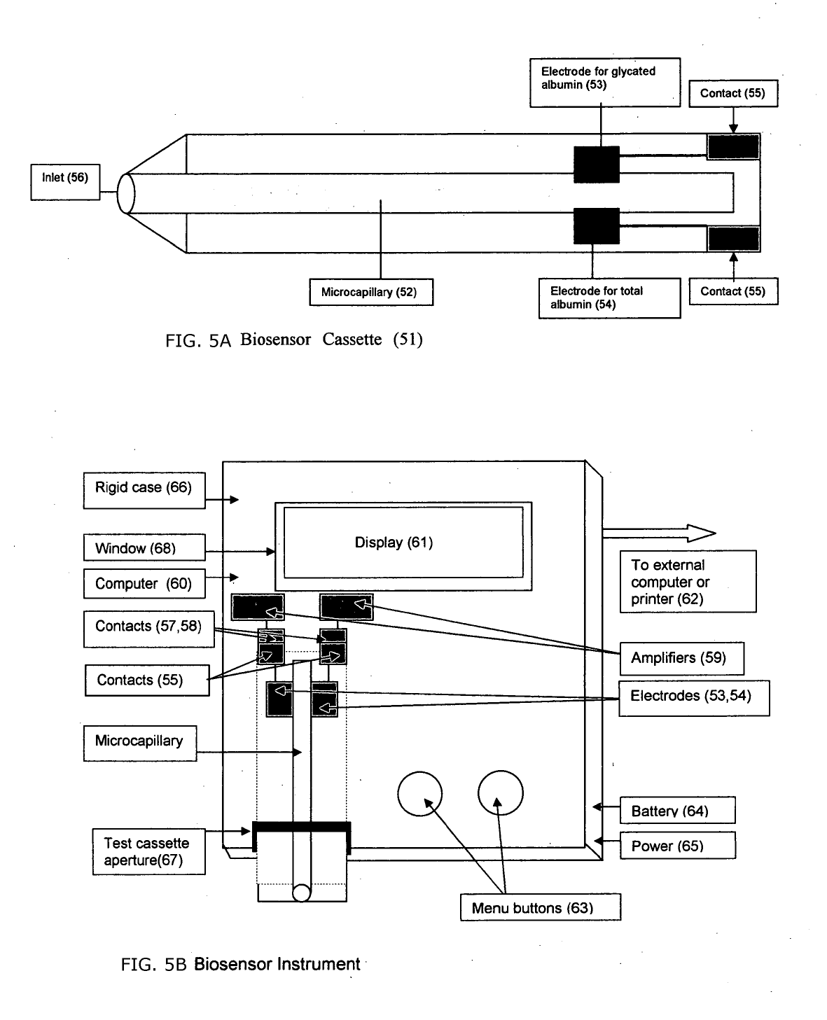

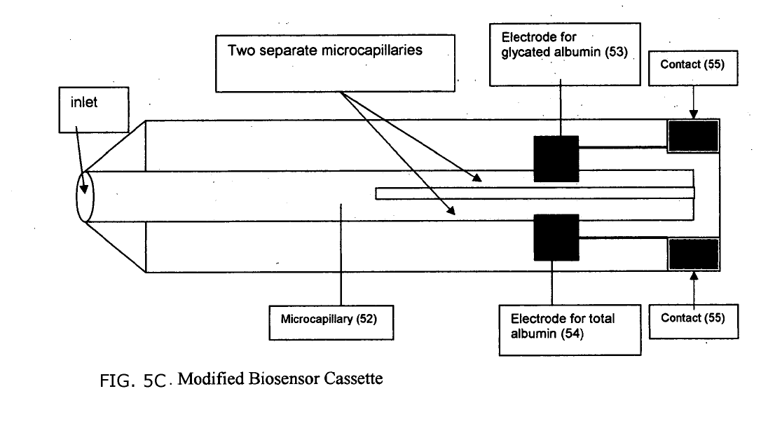

### DESCRIPTION OF THE INVENTION

This invention describes a procedure for measuring the percent of glycated albumin compared to total albumin in the patient's saliva. There are however, no published reports on the levels of glycated albumin and total albumin in saliva. The reason that there are no reference ranges for glycated albumin in saliva is because a) the levels of glycated albumin and total albumin in saliva are two orders of magnitude lower than the levels in plasma and b) the absolute concentration of glycated albumin and total albumin in saliva will vary according to the amount of saliva secreted. However, using a sensitive laboratory based immunoassay developed by this inventor it was found that the ratio of glycated albumin to total albumin in saliva is constant and shows a direct 1:1 correlation with glycated albumin to total albumin in plasma (unpublished experiments). This is not entirely unexpected because saliva albumin is made up of albumin from blood that has migrated thru the salivary glands and secreted as saliva. Albumin in blood has a circulating life of about 20 days and therefore the level of glycated albumin in blood represents the average glycated albumin level over approximately the preceding 2-3 weeks. As saliva albumin is derived from blood it will also provide an estimate of the average glycated albumin level over the preceding 2-3 week period. The assay procedure involves measuring glycated albumin and total albumin in the same sample simultaneously, and reporting the result as a ratio of glycated albumin to total albumin in the sample. This ratiometric method eliminates the requirement for a fixed, accurate volume of sample for testing, and also eliminates the need for internal standards as quantification of each individual analyte is not required. To perform the test the patient's saliva sample is placed in a test cassette that contains reagents to perform the test. The test cassette is then inserted into a measuring instrument that reads, calculates, stores and reports the result.

The novelty of this invention is that specific aptamers are used to detect the unique binding ligand(s) of glycated human albumin and also to detect the unique binding ligand(s) of human albumin. Current assay methods utilize monoclonal and polyclonal antibodies directed against human albumin and/or glycated human albumin. There are a number of drawbacks in using antibodies, and monoclonal antibodies in particular, as assay reagents. First, there are a very limited number of characterized monoclonal antibodies to glycated human albumin. Second, monoclonal antibodies are known to vary in their binding ability depending on the particular clone from which they were derived. Third, biological reagents such as antibodies are susceptible to deterioration when stored as components of kits, especially when these are stored at room temperature. Fourth, antibodies can only be produced to certain antigens that are recognized by the immunized host animal and the avidity and binding characteristics of the antibody will reflect the antigenicity of the antigen and the avidity of the antibody response. In contrast, the use of aptamers as described in this invention will overcome many of these limitations.

Aptamers are small (i.e., 40 to 100 bases), synthetic oligonucleotides (ssDNA or ssRNA) that can specifically recognize and bind to virtually any kind of target, including ions, whole cells, drugs, toxins, low-molecular-weight ligands, peptides, and proteins. Each aptamer has a unique configuration as a result of the composition of the nucleotide bases in the chain causing the molecule to fold in a particular manner. Because of their folded structure each aptamer will bind selectively to a particular ligand in a manner analogous to an antibody binding to its antigen.

The aptamers employed in this invention may be ssDNA and/or ssRNA. They may be synthesized as D-nucleotides and/or as L-nucleotides. Aptamers are usually synthesized from combinatorial oligonucleotide libraries using in vitro selection methods such as the Systematic Evolution of Ligands by Exponential Enrichment (SELEX). This is a technique used for isolating functional synthetic nucleic acids by the in vitro screening of large, random libraries of oligonucleotides using an iterative process of adsorption, recovery, and amplification of the oligonucleotide sequences. The iterative process is carried out under increasingly stringent conditions to achieve an aptamer of high affinity for a particular target ligand.

In this invention aptamers specific for glycated human albumin are synthesized using the SELEX method or other method of aptamer synthesis. Similarly, aptamers specific for human albumin are synthesized using the SELEX method or other method of aptamer synthesis. The specificity of the aptamers for their respective ligands are confirmed using enzyme-linked assays that are similar in principle to the enzyme-linked immunosorbent assay (ELISA).

Aptamers are synthesized and therefore their manufacture can be standardized with no variability in the end-product. This is in contrast to antibodies that have an inherent biological variability because of the way they are developed and purified. Aptamers are also more stable to degradation than antibodies and will bind to their respective ligand under conditions not possible with antibodies.

The equipment and procedures for synthesizing aptamers are commercially available and known to those skilled in the art. They are considered to be within the scope of this invention.

This invention describes several types of point-of-care devices for measuring glycated albumin using aptamers. One method is based on quantitative lateral flow chromatography and the other method is based on a biosensor device. All the devices however, have a common element, which is the use of aptamers as the key binding reagents in the assay. Also all methods use a ratiometric method for calculating and reporting the result.

A. Quantitative Lateral Flow Affinity Chromatography.

There are two components involved in the device: A membrane strip upon which the reagents for performing the test are disposed and a reading instrument that measures the results on the test strip. The test strip consists of a cellulose nitrate membrane or similar membrane support (1). There is a sample application pad (2) that serves to remove particulate material and allow the fluid component to flow through. Distal to the sample application pad there is a reaction pad containing a specific anti-albumin aptamer labeled with an indicator agent (3). Further along the membrane there is a band of anti-glycated albumin aptamer (4) fixed to the membrane; and further along the membrane there is a band of anti-albumin aptamer (5) fixed to the membrane. This membrane fixed anti-albumin aptamer has a different specificity from the aptamer labeled with the indicator agent so that binding of the indicator labeled aptamer to the albumin molecule will not block the capacity of the fixed anti-albumin aptamer to bind to the same albumin molecule at a different site. Further along the membrane there is a reservoir pad (6) at the distal end of the membrane. The test strip is enclosed within a rigid cassette (7) containing a sample well (8) for sample application and a measurement area (9) to allow for measurement of the test results using a measuring instrument such as a spectrometer, or a fluorometer, or a magnetic detector, or other measuring instrument.

The indicator agent may be fluorescent dyed latex beads, or colloidal gold particles, or paramagnetic particles. The anti-albumin aptamer can be attached to the indicator agent by adsorption or covalent binding to the surface of the indicator particles. These methods of attachment are known to those skilled in the art. And depending on the indicator agent used the measuring instrument will be a fluorometer, or spectrometer or magnetic assay reader.

Lateral Flow Affinity Chromatographic Procedure.

A saliva sample is placed in the sample well and allowed to absorb into the sample application pad. The sample application pad has a porosity that will filter out particulate material and allow the filtrate to flow through. In the preferred embodiment of this invention the test strip is enclosed in a cassette with an orifice at the sample application end so that the end of the strip can be placed within the mouth to collect the saliva sample directly.

The saliva sample migrates along the membrane and mixes with the indicator labeled anti-albumin aptamer reagent. The labeled reagent binds to the albumin present in the sample and the resultant complex migrates along the membrane until it contacts the band of fixed anti-glycated albumin aptamer. There the anti-glycated albumin aptamer will bind and fix any complex containing glycated albumin and in turn the indicator reagent moiety of the complex also becomes fixed. The remaining complexes that do not contain glycated albumin are not bound and continue to migrate along the membrane until they contact the band of fixed anti-albumin aptamer. There the anti-albumin aptamer will bind and fix the complexes containing albumin and in turn the indicator reagent also is fixed to the membrane.

The level of indicator agent bound to the membrane at the two reaction sites is measured using the appropriate measuring device. For example, if fluorescent beads are used then the measuring instrument is a fluorometer designed for this purpose; or if colloidal gold particles are used then the measuring instrument may be a reflectance spectrophotometer; or if paramagnetic particles are used the measuring instrument is a magnetic assay reader.

The intensity of the bands are directly proportional to the amount of glycated albumin and non-glycated albumin present in the saliva sample. The intensity of the bands are measured by the measuring instrument that also calculates the result according to a mathematical algorithm based on the formula:

Percentage ratio of glycated albumin compared to total albumin is:

$$\frac{A \times 100}{( A + B )}$$

where A is the glycated albumin band and B is the non-glycated albumin band.

The result is expressed as the percent of glycated albumin to total albumin and displayed on the instrument's display screen.

To monitor glycemic control the test is performed on a periodic basis and the results of successive testing are stored in the measuring instrument's memory. The results can be expressed as a numerical display and/or in a graphical format so that trend analysis of glycemic control over time can be performed. The results can also be sent to an external computer and/or printer and/or the internet for further storage and display.

Measuring Devices for Lateral Flow Assays.

The measuring devices share the same basic design. In the fluorometer (10) there is an excitation beam of light emitter (11) at the glycated albumin band with its corresponding fluorescence detector (12); and another excitation beam of light emitter (13) at the non-glycated albumin band with its corresponding fluorescence detector (14). The intensity of fluorescence from each band is measured and used to calculate the result. There is an on-board computer (15) that performs the calculations and reports the result, which is displayed on a liquid crystal display (16) or sent to an external computer or printer (17). Commands to the computer are made via a set of keys or menu buttons (18). The instrument is powered by a battery (19) or external power source (20). The external case is made of a rigid material (21) with an aperture (22) for insertion of the test cassette and a window (23) for the LCD.

The spectrophometer (24) used for measuring the intensity of color has a light source (25) to illuminate the glycated albumin band and a corresponding detector (26) to measure the color intensity of the glycated albumin band. There is a light source (27) to illuminate the non-glycated albumin band with its corresponding detector (28) to measure the color intensity of the non-glycated albumin band. The color intensity from each band is measured and used to calculate the result. There is an on-board computer (29) that performs the calculations and reports the result, which is displayed on a liquid crystal display (30) or sent to an external computer or printer (31). Commands to the computer are made via a set of keys or menu buttons (32). The instrument is powered by a battery (33) or external power source (34). The external case is made of a rigid material (35) with an aperture (36) for insertion of the test cassette and a window (37) for the LCD.

The magnetic assay reader (38) used for measuring the magnetic field changes has a series of magnetic detectors (39) to measure the magnetic field intensity at the site of the glycated albumin band (40) and at the site of the non-glycated albumin band (41). The magnet strength at each site is measured and used to calculate the result. There is an on-board computer (42) that performs the calculations and reports the result, which is displayed on a liquid crystal display (43) or sent to an external computer and/or printer (44) and/or the internet. Commands to the computer are made via a set of keys or menu buttons (45). The instrument is powered by a battery (46) or external power source (47). The external case is made of a rigid material (48) with an aperture (49) for insertion of the test cassette and a window (50) for the LCD.

Assay Materials:

The materials for this assay can be produced according to standard laboratory methods or purchased commercially. The membrane employed is a cellulose nitrate membrane or similar porous membrane.

The anti-glycated albumin aptamers are synthesized using the SELEX method or other methods of synthesis. The aptamers are attached to the membrane strip using standard laboratory procedures.

Alternatively, in one embodiment of this invention the anti-glycated albumin aptamer is replaced with chemicals known to bind glycated proteins such as phenyl boronic acids.

The anti-albumin aptamers are synthesized using the SELEX method or other methods of synthesis. It is important to note that the anti-albumin aptamer labeled with the indicator agent will have a different specificity from the anti-albumin aptamer fixed to the membrane. This will enable the fixed anti-albumin aptamer to bind to the albumin without being blocked by the binding of the indicator labeled anti-albumin aptamer to the same site on the albumin molecule.

The anti-albumin aptamer is labeled with either colloidal gold particles, or fluorescent dyed particles, or paramagnetic particles according to standard laboratory techniques that are familiar to those skilled in the art. For example, the anti-albumin aptamer is used to coat colloidal gold particles or fluorescent latex beads by physical adsorption or by covalent binding. The colloidal gold particles and latex particles are selected to have a specified diameter size within the size range of 5-100 nm. The paramagnetic particles are selected to have a specified diameter size within the size range of 5-500 nm.

These and other indicator labels for labeling the anti-albumin aptamer are known to those skilled in the art and are within the scope of this invention.

The general process for preparing lateral flow chromatographic assays are employed in this invention. These methods are known to those skilled in the art and do not affect the novelty of this invention which describes a method that utilizes aptamers for assessing glycemic control by measuring the ratio of glycated albumin to total albumin in a saliva, urine or other body fluid sample.

B. Biosensor Device

A biosensor is a device for the detection of an analyte that incorporates a biological component in the measurement process. The blood glucose meter is the most familiar example of a biosensor. There are different types of biosensors based on different types of physicochemical detector systems such as optical, piezoelectric, electrochemical, or magnetic.

Although aptamers are synthesized and not derived from biological material they are considered to be biomimic. Therefore a measuring device based on their use can be said to be a biosensor.

The biosensor of this invention consists of two components: a disposable biosensor cassette that contains the reactive reagents; and a reusable measuring biosensor device that measures, calculates, reports and records the result.

In the preferred embodiment of this invention a label-free electrochemical biosensor for measuring glycated albumin is described, but other biosensor methods may be similarly employed and are considered within the scope of this invention.

The biosensor cassette (51) consists of a microcapillary tube or microfluid container (52) that contains two aptamer modified electrodes (53,54). The aptamers are immobilized on the surface of the gold electrodes by self-assembly. One electrode has an aptamer that specifically binds to glycated albumin but not to non-glycated albumin (53). The other electrode has an aptamer that binds to albumin at a non-glycated site (54). Each electrode has an electronic contact surface (55) to contact with a corresponding contact surface in the biosensor measuring device. The cassette is made of rigid material with an inlet orifice (56) to permit entry of the test sample. In the preferred embodiment of this invention the inlet orifice of the biosensor cassette is placed within the mouth to collect the saliva sample directly.

The biosensor measuring device consists of a measuring and computerized device into which the cassette is inserted. Upon insertion of the cassette there is contact made between the electronic surface contacts of the cassette (55) with the corresponding electronic surface contacts of the measuring device (57,58). The measuring device will record and compute the background reading of the electronic sensors before any reaction has occurred. After insertion of the test cassette the measuring device will record and compute the actual test results minus the background reading. The measuring device incorporates amplifiers (59) responsible for adjusting the strength of the electronic signals, and a computer chip (60) for calculating and storage of the result which is displayed on a liquid crystal display (61) or sent to an external computer and/or printer (62) and/or the internet. Commands to the computer are made via a set of keys or menu buttons (63). The instrument is powered by a battery (64) or external power source (65). The external case is made of a rigid material (66) with an aperture (67) for insertion of the test cassette and a window (68) for the LCD.

The test is performed as follows: When saliva or other fluid sample comes into contact with the inlet orifice the sample is drawn into the microcapillary container by capillary action. The sample then migrates along the microcapillary container until it contacts the aptamer coated electrodes. When this occurs the anti-glycated albumin aptamer will bind to glycated albumin in the sample causing a change in the electric potential of the anti-glycated aptamer electrode with the change being proportional to the amount of glycated albumin bound. Similarly, the anti-albumin aptamer will bind to albumin in the sample causing a change in the electric potential of the anti-albumin aptamer electrode with the change being proportional to the amount of albumin bound. [0060] The ratio of glycated albumin to total albumin can then be calculated as follows:

Percentage ratio of glycated albumin compared to total albumin is:

$$\frac{A \times 100}{B}$$

where A is the glycated albumin measurement and B is the albumin measurement.

In another embodiment of this invention the inlet microcapillary in the biosensor cassette is split into two microfluidic streams in the vicinity of the electrodes so that each aptamer will only bind to its respective ligand (5c). This separation of the sample into two streams ensures that the anti-glycated albumin aptamer electrode will only measure glycated albumin in its stream and the anti-albumin aptamer electrode will measure total albumin in its stream. The ratio of glycated albumin to total albumin can then be calculated as follows:

Percentage ratio of glycated albumin compared to total albumin is:

$$\frac{A \times 100}{B}$$

where A is the glycated albumin measurement and B is the albumin measurement.

The result is expressed as the percent of glycated albumin to total albumin and displayed numerically and graphically on the instrument's display screen. To monitor glycemic control the test is performed on a periodic basis and the results of successive testing are stored in the measuring instrument's memory. The results can be expressed as a numerical display and/or in a graphical format so that trend analysis of glycemic control over time can be performed. The results can also be sent to an external computer and/or printer and/or the internet for further storage and display.

From the description of this invention it is obvious that the same test system utilizing aptamers to measure glycated albumin in saliva can also be used to measure glycated albumin in other body fluids such as blood, or serum or plasma or urine.

In a further embodiment of this invention other analytes present in the sample may be simultaneously measured using the same procedure and measuring device. For example, in monitoring kidney function in diabetes it is useful to measure the amount of albumin in urine relative to a reference protein such as creatinine that is also present in the same urine sample. The biosensor device described in this invention for measuring glycated and total albumin can be adapted to have a third electrode sensor that is coated with an anti-creatinine aptamer for measuring the level of creatinine. The biosensor device can be designed as either a single microcapillary with a varying number of electrodes embedded at different locations within the microcapillary or the inlet microcapillary may be divided into two or more microcapillaries each with an electrode for measuring a different analyte. The results are reported as the ratio of albumin in urine relative to the amount of creatinine in the same urine sample. At the same time the ratio of glycated albumin relative to total albumin can also be reported for the same sample.

In this invention the measuring instrument for the lateral flow device and the biosensor is designed to be small, portable, and user-friendly. It will look and feel similar to the glucose meters that are in common use.

The description and examples presented in this invention are gives as illustration and not as limitation. Those of ordinary skill in the art will recognize from the description and examples given in this invention other embodiments and applications that fall within the spirit and scope of this invention.

1.-7. (canceled)

8. A method for measuring glycated albumin in a sample comprising:
contacting said sample with a biosensor cassette;
contacting said sample within said biosensor cassette with anti-glycated albumin aptamer and an anti-albumin aptamer;
placing said sample-containing biosensor casette into a reusable measuring instrument; and
determining the ratio of glycated albumin to total albumin in the sample.

9. The method for measuring glycated albumin according to claim 8 wherein the biosensor cassette comprises a microfluid container which comprises an inlet orifice for placement of said sample; an anti-glycated albumin aptamer coated electrode; and an anti-albumin aptamer coated electrode.

10. The method according to claim 8 wherein the measuring instrument is a biosensor instrument comprising a first electronic sensor for measuring the glycated albumin bound to the anti-glycated albumin apatamer-coated electrode; a second electronic sensor for measuring albumin bound to the anti-albumin aptamer-coated electrode; an internal computer for measurement and calculation; a liquid crystal display; an external port to transfer data to an external computer and/or printer and/or the internet; a battery and/or an external power source; and a rigid external case with an aperture for inserting the biosensor cassette.

11. The method according to claim 8 wherein the measuring instrument yields a plurality of test results obtained from testing the same individual over a period of time and wherein said test results are stored in the measuring instrument's computer memory.

12. The method according to claim 8 wherein the sample for testing is selected from the group consisting of saliva, urine, and blood.

13. The method according to claim 11 wherein the stored data is retrievable on demand.

14. The method according to claim 11 wherein the test results are expressed in a numerical format and/or in a graphical format.

15. The method according to claim 11 wherein the test results are displayed on the instrument's display monitor and/or transferred to an external computer or printer and/or the internet.

16. The method according to claim 9 wherein the microfluid container is a microcapillary tube

17. A method for measuring glycated albumin in a sample comprising the steps of:
contacting the sample with an inlet orifice of a biosensor cassette;
drawing the sample from the inlet orifice into at least one microfluid container within said biosensor cassette;
contacting the sample with an anti-glycated albumin aptamer-coated electrode and an anti-albumin aptamer-coated electrode thereby changing the electric potential of the electrode;
placing said sample-containing biosensor cassette into a reusable measuring instrument; and
determining the ratio of glycated albumin to total albumin in the sample based on the change in electric potential of the electrodes.
